(19) United States
(12) Patent Application Publication
(10) Pub. No.: US 2017/0020459 A1
(43) Pub. Date: Jan. 26, 2017

(54) MOTION COMPENSATED BIOMEDICAL SENSING

(71) Applicant: Edwards Lifesciences Corporation, Irvine, CA (US)

(72) Inventor: Feras Al Hatib, Irvine, CA (US)

(73) Assignee: Edwards Lifesciences Corporation, Irvine, CA (US)

(21) Appl. No.: 15/215,091

(22) Filed: Jul. 20, 2016

Related U.S. Application Data
(60) Provisional application No. 62/195,734, filed on Jul. 22, 2015.

(51) Int. Cl. A61B 5/00; A61B 5/0205

(52) U.S. Cl. CPC A61B 5/721; A61B 5/743; A61B 5/681; A61B 5/725; A61B 5/0205; A61B 5/6804; A61B 5/0002

(57) ABSTRACT

There is provided a biomedical sensing system and a method for its use. Such a system includes a diagnostic sensor configured to sense a physiological metric of a living subject via contact with the living subject, and to generate a diagnostic signal corresponding to the physiological metric. In addition, the system includes a motion sensor situated proximate the diagnostic sensor and configured to sense a motion corresponding to a motion of the diagnostic sensor during sensing of the physiological metric. The system also includes an analysis unit including a processor, and a memory storing a motion correction module. The processor is configured to receive the diagnostic signal and the motion signal, and to execute the motion correction module from the memory to adaptively filter the diagnostic signal using the motion signal to produce a motion compensated diagnostic signal.

### BACKGROUND

The sensing and monitoring of vital human physiological processes, such as the measurement of respiration, pulse rate, body temperature, and other vital signs, form an important part of effective medical diagnosis and treatment. Traditionally, vital signs and other human physiological metrics of interest have been measured periodically, in a controlled clinical setting, and under circumstances in which a patient may be substantially immobile.

However, in some instances, the patient may derive greater benefit from a sustained and substantially continuous monitoring of one or more physiological metrics during patient interaction with a normal home and/or work environment. One conventional approach to monitoring cardiac function, for example, over an extended period outside of a clinical setting includes outfitting the patient with a harness including multiple electrodes attached to the chest, abdomen, and back, and requires the patient to wear the harness for hours or days while engaging in normal activity. Although the data derived from use of this approach may be of significant diagnostic value, the experience of wearing such a harness and electrode arrangement is typically at least inconvenient, and may be uncomfortable and/or upsetting to the patient.

### SUMMARY

There are provided systems and methods for performing motion compensated biomedical sensing, substantially as shown in and/or described in connection with at least one of the figures, and as set forth more completely in the claims.

### BRIEF DESCRIPTION OF THE DRAWINGS

FIG. 1 shows a diagram of an exemplary system for performing motion compensated biomedical sensing, according to one implementation;

FIG. 2 shows a diagram of an exemplary system for performing motion compensated biomedical sensing, according to another implementation;

FIG. 3A shows a top view of an exemplary wearable article suitable for use as part of a system for performing motion compensated biomedical sensing, according to one implementation;

FIG. 3B shows a cross-sectional view of the exemplary wearable article shown in FIG. 3A;

FIG. 3C shows an exemplary wearable article suitable for use as part of a system for performing motion compensated biomedical sensing, according to another implementation;

FIG. 3D shows the exemplary wearable article shown in FIG. 3C in the form of a cuff configured to encircle a human digit, such as a finger, according to one implementation;

FIG. 3E shows an exemplary system for performing motion compensated biomedical sensing and integrated with a wearable article, according to one implementation;

FIG. 3F shows an exemplary system for performing motion compensated biomedical sensing and integrated with a wearable article, according to another implementation; and

FIG. 4 is a flowchart presenting an exemplary method for performing motion compensated biomedical sensing, according to one implementation.

### DETAILED DESCRIPTION

The following description contains specific information pertaining to implementations in the present disclosure. One skilled in the art will recognize that the present disclosure may be implemented in a manner different from that specifically discussed herein. The drawings in the present application and their accompanying detailed description are directed to merely exemplary implementations. Unless noted otherwise, like or corresponding elements among the figures may be indicated by like or corresponding reference numerals. Moreover, the drawings and illustrations in the present application are generally not to scale, and are not intended to correspond to actual relative dimensions.

The present disclosure provides sensing equipment that can be small, lightweight, comfortable, and minimally intrusive upon normal patient movement and activity. Moreover, the present disclosure advantageously provides sensing equipment that is wearable by a person in such a way that the person's awareness of the sensing equipment is substantially minimized. However, the very freedom of movement and unselfconsciousness enabled by such design may, if not properly compensated for, introduce motion generated noise artifacts into the physiological metric or metrics being monitored by the sensing equipment. For example, a small, comfortable biomedical sensing system worn on an extremity of the person may undergo frequent, rapid movement through a wide range of motion as the person moves his or her arms or legs during normal activity.

The present application discloses systems and methods for performing motion compensated biomedical sensing. According to the various implementations disclosed herein, a motion sensor situated in proximity to one or more diagnostic sensors is utilized to sense motion of the diagnostic sensor or sensors as diagnostic sensing is performed. As discussed in greater detail below, a motion signal received by an analysis unit of a biomedical sensing system can be used to filter a diagnostic signal received from the diagnostic sensor(s) that corresponds to a physiological metric of a living subject. Such filtering of the diagnostic signal based on the motion signal produces a motion compensated diagnostic signal that has been corrected for the motion of the diagnostic sensor(s) during sensing. The analysis unit may then determine a measurement of the physiological metric being sensed that is substantially accurate despite the motion of the diagnostic sensor(s) during sensing.

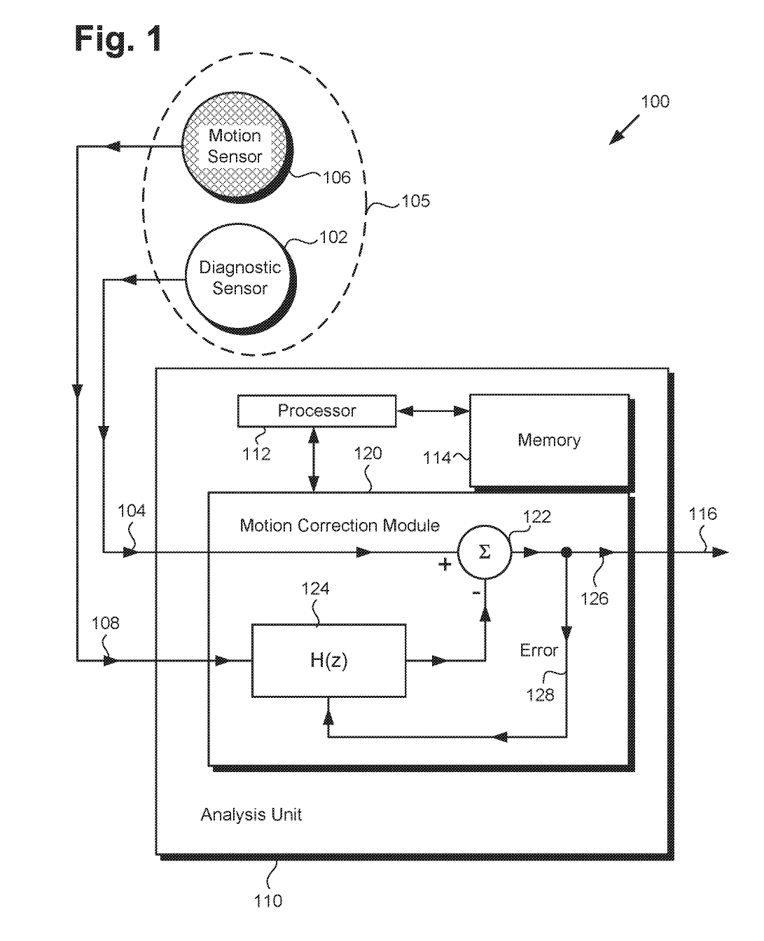

FIG. 1 shows a diagram of an exemplary system for performing motion compensated biomedical sensing, according to one implementation. As shown in FIG. 1, biomedical sensing system 100 includes diagnostic sensor 102, motion sensor 106 situated in proximity to diagnostic sensor 102, and analysis unit 110 including processor 112, memory 114, and motion correction module 120 stored by memory 114. As further shown in FIG. 1, analysis unit 110 is configured to receive diagnostic signal 104 from diagnostic sensor 102 and motion signal 108 from motion sensor 106, and to determine measurement 116 of a physiological metric being sensed by diagnostic sensor 102. Also shown in FIG. 1 are dashed proximity boundary 105, summation block or “summer” 122, signal processing block 124, and error signal 128 of motion correction module 120, as well as motion compensated diagnostic signal 126 produced by motion correction module 120 using summer 122, signal processing block 124, and error signal 128.

It is noted that although FIG. 1 depicts diagnostic sensor 102 as being implemented using a single sensor, that representation is provided merely as an aid to conceptual clarity. More generally, diagnostic sensor 102 may correspond to one or more diagnostic sensors used in combination to sense a physiological metric of interest. For example, diagnostic sensor or sensors 102 may be utilized to sense the blood pressure, cardiac rhythm, or blood chemistry, such as blood glucose or oxygen saturation, of a human or a non-human animal subject with whom or which diagnostic sensor 102 makes non-invasive epidermal contact. It is further noted that although FIG. 1 depicts motion sensor 106 as being implemented using a single sensor, motion sensor 106, like diagnostic sensor 102, may be implemented using one or more sensors.

Motion sensor 106 is situated proximate diagnostic sensor 102, as shown by dashed proximity boundary 105, in order to sense the motion of diagnostic sensor 102 during sensing. The specific dimensions of proximity boundary may vary depending upon the placement of diagnostic sensor 102 on the subject, the size of the subject, the physiological metric being sensed by diagnostic sensor 102, and/or the sensing environment in which the sensing is performed, for example. For the purposes of the present disclosure, situating motion sensor 106 “proximate” diagnostic sensor 102 is to be construed as situating motion sensor 106 so as to cause motion sensor 106 to experience substantially the same motion experienced by diagnostic sensor 102 during sensing.

Processor 112 may be a hardware central processing unit (CPU) for biomedical sensing system 100, for example, in which role processor 112 executes the software and/or firmware utilized by analytical unit 110, executes motion correction module 120, and controls the transfer of data to and from memory 114. Motion correction module 120 may be implemented as a filter configured to filter diagnostic signal 104 based on motion signal 108, and to produce motion compensated diagnostic signal 126. According to the implementation shown in FIG. 1, diagnostic signal 104 may include a diagnostic signal component and a noise artifact component resulting from motion of diagnostic sensor 102 during sensing. Motion compensated diagnostic signal 126 may provide a substantially accurate representation of the diagnostic component of diagnostic signal 104. That is to say, motion compensated diagnostic signal 126 is corrected for the motion of diagnostic sensor 102 during sensing through removal of the noise artifact component of diagnostic signal 104 by motion correction module 120.

As shown by FIG. 1, in some implementations, motion correction module 120 may be configured as an adaptive filter having a closed loop configuration. An adaptive filter can be implemented using a linear filter having a transfer function controlled by one or more variable parameters, and a mechanism for adjusting the parameter or parameters according to an optimization algorithm. That functionality is represented in FIG. 1 by signal processing block 124 having adjustable transfer function H(z). The closed loop configuration of motion correction module 120 enables use of error signal 128 as a feedback signal for adjusting transfer function H(z). As a result, signal processing block 124 and summer 122 can be implemented so as to filter diagnostic signal 104 using motion signal 108, to effectively filter out an undesired noise artifact component present in diagnostic signal 104, to produce motion compensated diagnostic signal 126.

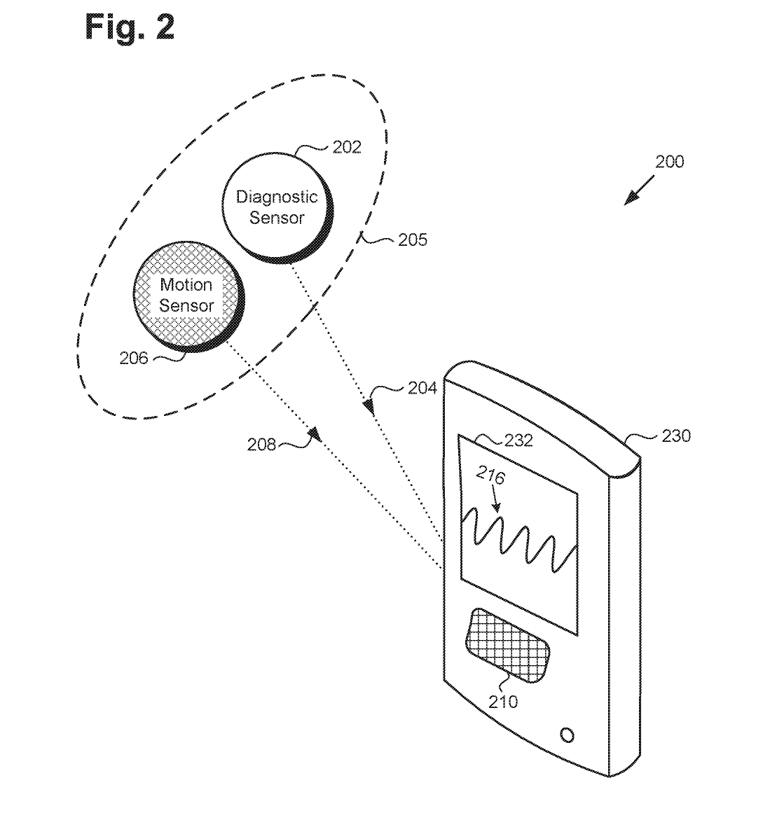

Referring now to FIG. 2, FIG. 2 shows a diagram of an exemplary system for performing motion compensated biomedical sensing, according to another implementation. Biomedical sensing system 200 includes diagnostic sensor 202, motion sensor 206 situated in proximity to diagnostic sensor 202, and analysis unit 210 implemented as part of personal communication device 230 including display 232. As shown by the dotted lines linking diagnostic sensor 202 and motion sensor 206 to personal communication device 230, according to the implementation shown in FIG. 2, one or both of diagnostic signal 204 and motion signal 208 may be transmitted wirelessly or via wires to personal communication device 230. Analysis unit 210 is designed to receive diagnostic signal 204 and motion signal 208 and to determine a measurement of a subject physiological metric being sensed by diagnostic sensor 202. Also shown in FIG. 2 is proximity boundary 205 defining a boundary for placement of motion sensor 206 close to diagnostic sensor 202, and waveform 216 corresponding to the sensed physiological metric and displayed on display 232 of personal communication device 230.

Diagnostic sensor 202, diagnostic signal 204, motion sensor 206, motion signal 208, and proximity boundary 205 correspond in general to respective diagnostic sensor 102, diagnostic signal 104, motion sensor 106, motion signal 108, and proximity boundary 105, in FIG. 1, and may share any of the characteristics attributed to those corresponding features, above. In addition, analysis unit 210, in FIG. 2, corresponds in general to analysis unit 110, in FIG. 1, and may share any of the characteristics attributed to analysis unit 110, above. In other words, although not shown as such in FIG. 2, analysis unit 210 includes a motion correction module corresponding in general to motion correction module 120, in FIG. 1. Moreover, the motion correction module of analysis unit 210, in FIG. 2, includes a summer and a signal processing blocking, and is configured to produce a motion compensated diagnostic signal, corresponding respectively to summer 122, signal processing block 124, and motion compensated diagnostic signal 126, in FIG. 1.

As noted above, diagnostic sensor 102/202 is configured for contact with a living subject, which may be a human subject or a non-human animal subject. Referring to a human subject merely for exemplary purposes, diagnostic sensor 102/202 may be placed so as to make contact with a finger, toe, wrist, ankle, forearm, or lower leg of the subject, for example. As also noted above, motion sensor 106/206 is situated proximate diagnostic sensor 102/202 in order experience substantially the same motion experienced by diagnostic sensor 102/202 during sensing by diagnostic sensor 102/202. However, in contrast to diagnostic sensor 102/202, motion sensor 106/206 may or may not be in contact with or touch the subject. For example, where diagnostic sensor 102/202 makes contact with a wrist or ankle of a subject, motion sensor 106/206 may also make contact with or touch the subject's wrist or ankle, or may be attached to a wearable article including the diagnostic sensor, or to an adjacent portion of a clothing item of the subject, such as a shirt sleeve, stocking, or pant leg, without contact with or touching the subject.

According to the implementation shown by FIG. 2, analysis unit 210 is implemented as part of personal communication device 230, which is depicted as a smartphone in FIG. 2. In such an implementation, personal communication device 230 may be a component of biomedical sensing system 200, and the processor and memory resources of personal communication device 230 may be utilized so as to correspond respectively to processor 112 and memory 114, in FIG. 1. Moreover, according to the implementation shown in FIG. 2, motion correction module 120 may be an application that can be downloaded to memory 114 of personal communication device 230 for execution by processor 112 to generate waveform 216 on display 232.

Waveform 216 corresponds to measurement 116 of the physiological metric determined by analysis unit 110, in FIG. 1. Waveform 216 may correspond to a cardiac rhythm, fluctuations in blood pressure or blood chemistry, for example, or to any other variable physiological metric of diagnostic interest. It is noted that although personal communication device 230 is shown as a smartphone in FIG. 2, that representation is provided merely as an example. In other implementations, personal communication device 230 may be a personal communication device other than a smartphone, such as a digital media player, a laptop or desktop personal computer (PC), a tablet computer, a smartwatch, or any other computing device.

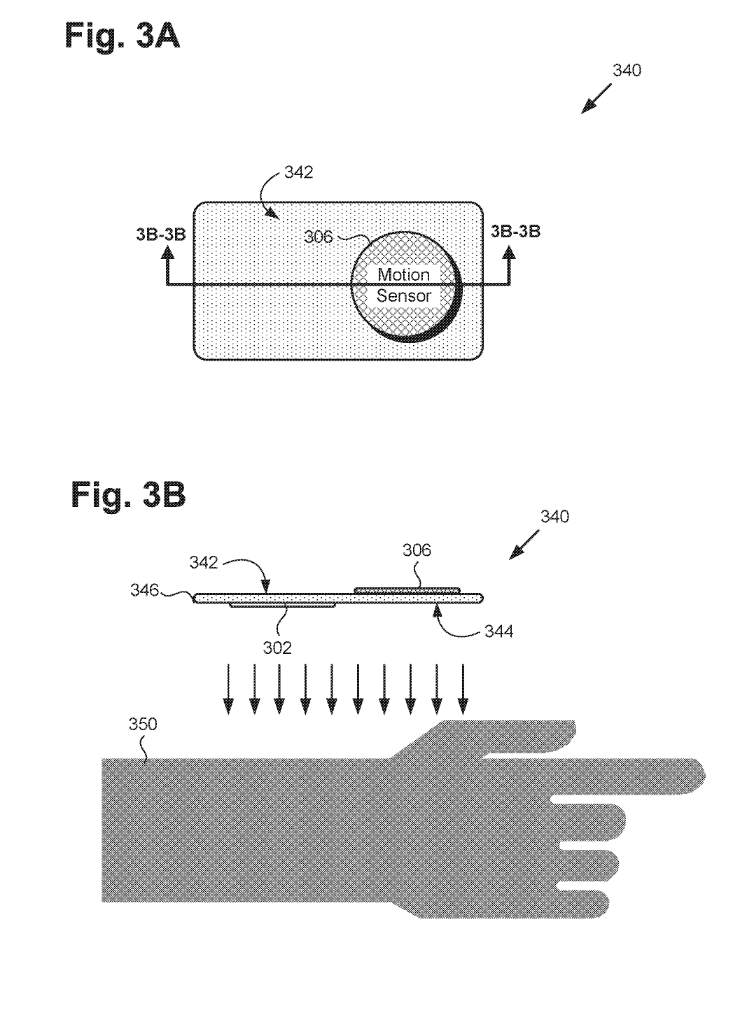

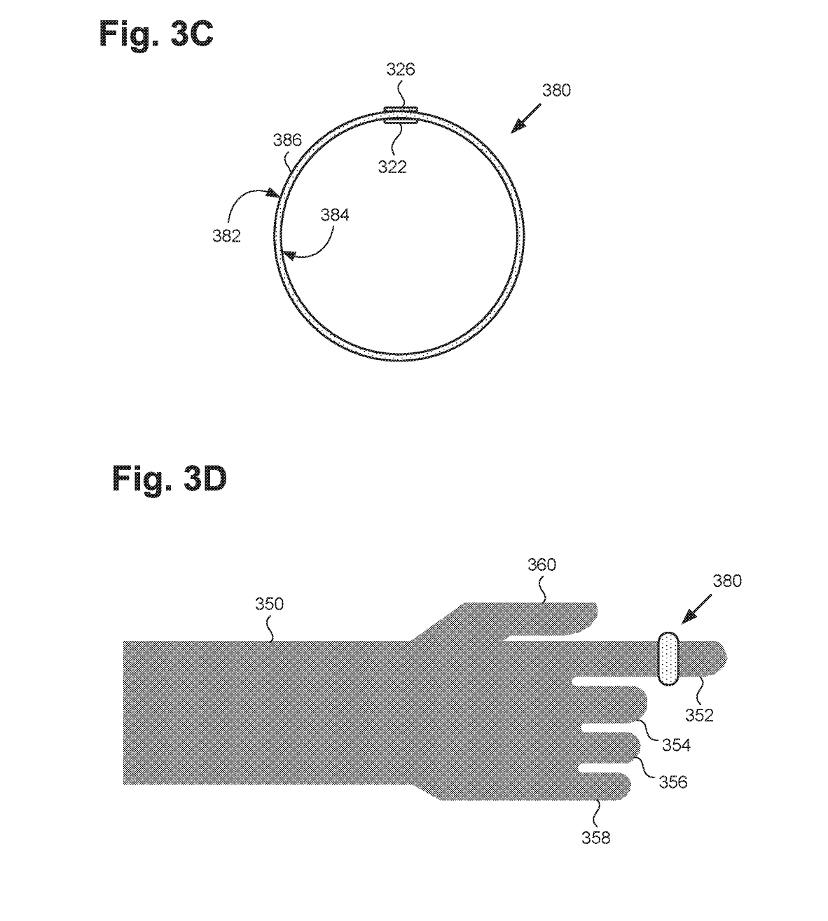

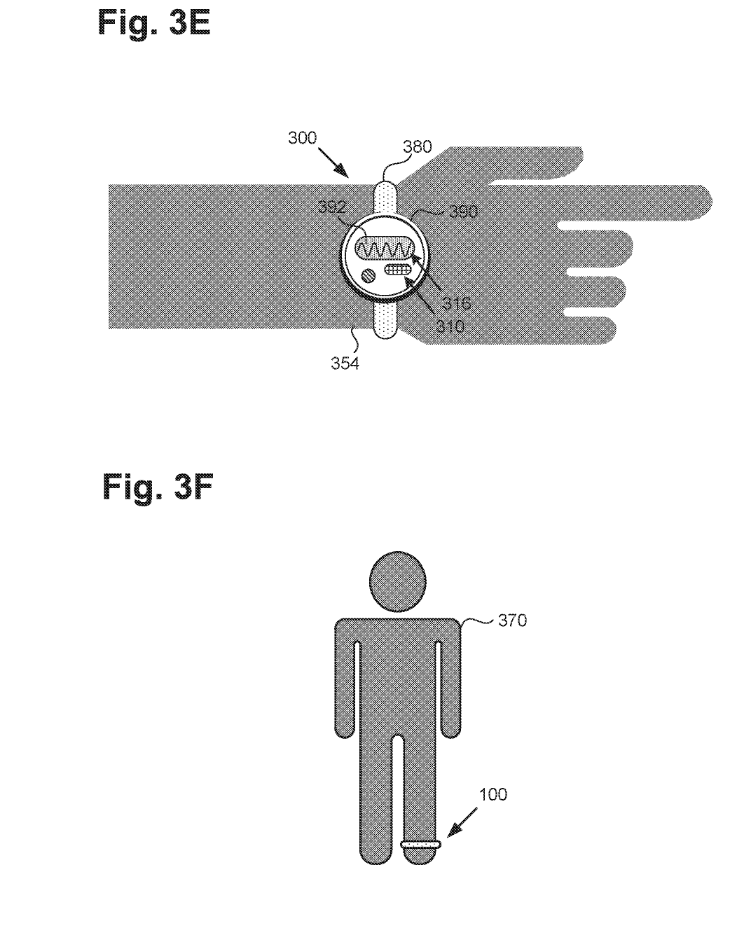

Moving to FIGS. 3A and 3B, FIG. 3A shows a top view of exemplary wearable article 340 suitable for use as part of a system for performing motion compensated biomedical sensing, according to one implementation. FIG. 3B shows a cross-sectional view of wearable article 340 along perspective lines 3B-3B in FIG. 3A. Wearable article 340 includes material 346, which may be formed of a natural or synthetic fabric, or a natural or synthetic polymeric material, such as rubber or polyurethane, for example.

As shown in FIGS. 3A and 3B, material 346 of wearable article 340 has top surface 342 and bottom surface 344, which may be an adhesive surface, for example, opposite top surface 342. As further shown in FIGS. 3A and 3B, wearable article 340 includes diagnostic sensor 302 affixed to bottom surface 344 of material 346, and includes motion sensor 306 affixed to top surface 342 of material 346. Diagnostic sensor 302 and motion sensor 306 correspond in general to respective diagnostic sensor 102/202 and motion sensor 106/206 in FIGS. 1 and 2, and may share any of the characteristics attributed to those corresponding features, above.

According to the implementation shown by FIGS. 3A and 3B, wearable article 340 may take the form of a patch, such as an adhesive patch, for example, designed for epidermal attachment to human arm 350, shown to include a portion of a forearm, wrist, and hand in FIG. 3B. As a result, wearable article 340 is designed to place diagnostic sensor 302, which is configured for sensing through non-invasive epidermal contact with arm 350, in contact with an epidermal surface of arm 350. Moreover, and as shown in FIG. 3B, wearable article 340 includes material 346 situated between diagnostic sensor 302 and motion sensor 306. Material 346 may have sufficient dielectric properties to electrically isolate motion sensor 306 from diagnostic sensor 302, while allowing motion sensor 306 to be situated proximate diagnostic sensor 302.

It is noted that although arm 350 is depicted as a distal portion of a human arm in FIG. 3B, arm 350 may correspond more generally to either a proximal or distal portion of a human limb, and may be a portion of an arm or a leg. Furthermore, and as discussed more specifically by reference to FIG. 3C, FIG. 3D, and FIG. 3E, below, in other implementations, wearable article 340 may take the form of a ring, band, or cuff, rather than the patch implementation shown in FIGS. 3A and 3B.

Referring to FIG. 3C, FIG. 3C shows wearable article 380 according to another exemplary implementation. As shown in FIG. 3C, wearable article 380 may assume a ring or band shape having outer surface 382 and inner surface 384. As further shown in FIG. 3C, wearable article 380 includes diagnostic sensor 322 affixed to inner surface 384, and includes motion sensor 326 affixed to outer surface 382. Diagnostic sensor 322 and motion sensor 326 correspond in general to respective diagnostic sensor 102/202/302 and motion sensor 106/206/306 in FIGS. 1, 2, 3A, and 3B, and may share any of the characteristics attributed to those corresponding features, above.

According to the implementation shown by FIG. 3C, wearable article 380 is designed to encircle a human appendage, for example a wrist or ankle, or a digit such as a finger or toe. As a result, wearable article 380 is designed to place diagnostic sensor 322, which is configured for sensing through non-invasive epidermal contact with a living subject, in contact with an epidermal surface of the subject. Moreover, and like the implementation shown in FIGS. 3A and 3B, material 386 of wearable article 380 in FIG. 3C is situated between diagnostic sensor 322 and motion sensor 326. Similar to material 346, material 386 may have sufficient dielectric properties to electrically isolate motion sensor 326 from diagnostic sensor 322, while allowing motion sensor 326 to be situated proximate diagnostic sensor 322.

Continuing to FIG. 3D, FIG. 3D shows exemplary wearable article 380 of FIG. 3C in the form of a cuff designed to encircle a human digit, such as index finger 352, according to one implementation. FIG. 3D shows arm 350 including thumb 360, index finger 352, middle finger 354, ring finger 356, and little finger 358. Although not visible in the perspective shown in FIG. 3D, wearable article 380 includes diagnostic sensor 322 and motion sensor 326 arranged in a configuration similar to that shown and described by reference to FIG. 3C, above.

According to the implementation shown in FIG. 3D, wearable article 380 is designed to place diagnostic sensor 322, which is configured for sensing through non-invasive epidermal contact with a finger, such as index finger 352, in contact with the finger. It is noted that although wearable article is shown as being worn on index finger 352, in FIG. 3D, that representation is provided merely by way of example. In other implementations, wearable article 380 may be worn on any of thumb 360, middle finger 354, ring finger 356, or little finger 358. Moreover, in some implementations, wearable article 380 may be adapted so as to be worn on a toe.

Referring now to FIG. 3E, FIG. 3E shows an exemplary system for performing motion compensated biomedical sensing that is integrated with a wearable article, according to one implementation. As shown in FIG. 3E, wearable smartwatch 300 includes wearable article 380 in the form of a wristband and smartwatch 390 affixed to wearable article 380. As further shown in FIG. 3E, smartwatch 390 includes analysis unit 310 of wearable smartwatch 300, and is designed to determine waveform 316 corresponding to a physiological metric being sensed by wearable smartwatch 300, and to display waveform 316 on display 392 of wearable smartwatch 300.

Wearable smartwatch 300 including analysis unit 310 corresponds in general to biomedical sensing system 100/200 including analysis unit 110/210, in FIGS. 1 and 2, and may share any of the characteristics attributed to those corresponding features, above. That is to say, although not shown as such in FIG. 3E, wearable smartwatch 300 includes diagnostic sensor 322 and motion sensor 326 shown in FIG. 3C. In addition, although also not shown in FIG. 3E, analysis unit 310 includes a motion correction module corresponding to motion correction module 120 in FIG. 1. It is noted that the motion correction module of analysis unit 310, in FIG. 3E, includes a summer and a signal processing blocking, and is configured to produce a motion compensated diagnostic signal, corresponding respectively to summer 122, signal processing block 124, and motion compensated diagnostic signal 126, in FIG. 1.

According to the implementation shown in FIG. 3E, the processor and memory resources of smartwatch 390may be utilized so as to correspond respectively to processor 112 and memory 114, in FIG. 1. As shown in FIG. 3E, smartwatch 390 with which wearable article 380 is integrated, is designed to be worn around wrist 354. As a result, wearable smartwatch 300 is designed to place diagnostic sensor 322, which is configured for sensing through non-invasive epidermal contact with a human, in contact with an epidermal surface of wrist 354. Referring to FIG. 3F, it is noted that, in another implementation, biomedical sensing system 100 can be adapted to be worn around an ankle or lower leg of subject 370, shown merely for exemplary purposes as human subject 370.

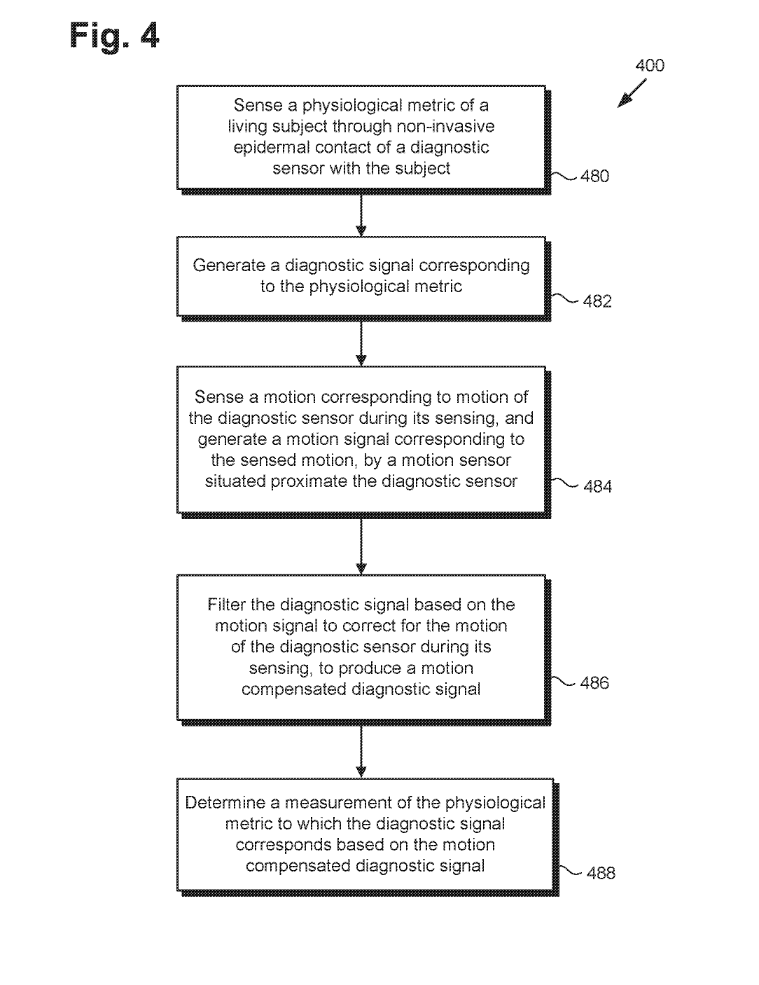

Referring to FIG. 4 in combination with FIGS. 1, 2, 3A, 3B, 3C, 3D, 3E, and 3F, flowchart 400 begins with sensing a physiological metric of a living subject through non-invasive epidermal contact of a diagnostic sensor with the subject (action 480). Sensing of the physiological metric may be performed by biomedical sensing system 100/200/300, using diagnostic sensor 102/202/302/322. As discussed above, diagnostic sensor 102/202/302/322 may correspond to one or more diagnostic sensors used in combination to sense a human physiological metric, or a physiological metric of a non-human animal. For example, diagnostic sensor 102/202/302/322 may be designed for non-invasive epidermal contact with a skin surface of the subject covering an artery or capillary of the subject.

Flowchart 400 continues with generating diagnostic signal 104/204 corresponding to the physiological metric (action 482). Diagnostic signal 104/204 may be generated by biomedical sensing system 100/200/300, using diagnostic sensor 102/202/302/322. Diagnostic sensor 102/202/302/322 may be configured to sense the pulse, blood pressure, or blood chemistry, for example, of a living subject through non-invasive epidermal contact with the subject. Diagnostic sensor 102/202/302/322 may be further configured to transform that sensed physiological metric into diagnostic signal 104/204, which may be a digital signal, for example, and to transmit diagnostic signal 104/204 to analysis unit 110/210/310. Moreover, in various implementations, diagnostic sensor 102/202/302/322 may be configured to generate diagnostic signal 104/204 and to transmit diagnostic signal 104/204 wirelessly or via wires.

Flowchart 400 continues with sensing a motion corresponding to motion of diagnostic sensor 102/202/302/322 during its sensing, and generating motion signal 108/208 corresponding to the sensed motion, by motion sensor 106/206/306/326 situated proximate diagnostic sensor 102/202/302/322 (action 484). Motion sensor 106/206/306/326 may include an accelerometer, for example, and may be configured to transform the sensed motion of diagnostic sensor 102/202/302/322 into motion signal 108/208. Motion sensor 106/206/306/326 is further configured to transmit motion signal 108/208, which may be a digital signal, for example, to analysis unit 110/210/310.

Analogously to diagnostic sensor 102/202/302/322, in various implementations, motion sensor 106/206/306/326 may be configured to generate motion signal 108/208 and to transmit motion signal 108/208 wirelessly or via wires. Moreover, and as discussed above, situating motion sensor 106/206/306/326 proximate diagnostic sensor 102/202/302/322 is to be construed as situating motion sensor 106/206/306/326 relative to diagnostic sensor 102/202/302/322 so as to cause motion sensor 106/206/306/326 to experience substantially the same motion experienced by diagnostic sensor 102/202/302/322 during sensing.

Flowchart 400 continues with filtering diagnostic signal 104/204 based on motion signal 108/208 to correct for the motion of diagnostic sensor 102/202/302/322 during its sensing, to produce motion compensated diagnostic signal 126 (action 486). Filtering of diagnostic signal 104/204 based on motion signal 108/208 may be performed by analysis unit 110/210/310 of biomedical sensing system 100/200/300, using motion correction module 120. As discussed above, motion correction module 120 may be implemented as a filter configured to filter diagnostic signal 104/204 based on motion signal 108/208, and to produce motion compensated diagnostic signal 126. Moreover, in some implementations, motion correction module 120 may be configured to utilize summer 122, signal processing block 124, and error signal 128 to perform adaptive filtering of diagnostic signal 104/204 based on motion signal 108/208. That is to say, in some implementations, motion correction module 120 may be configured to function as an adaptive filter of analysis unit 110/210/310.

Diagnostic signal 104/204 generated by diagnostic sensor 102/202/302/322 may include a diagnostic signal component and a noise artifact component resulting from motion of diagnostic sensor 102/202/302/322 during sensing. Motion compensated diagnostic signal 126 provides a substantially accurate representation of the diagnostic component of diagnostic signal 104/204, while substantially omitting the noise artifact component. That is to say, motion compensated diagnostic signal 126 is corrected for the motion of diagnostic sensor 102/202/302/322 during sensing through removal of the noise artifact component of diagnostic signal 104/204 by motion correction module 120.

Flowchart 400 concludes with determining measurement 116 of the physiological metric to which diagnostic signal 104/204 corresponds based on motion compensated diagnostic signal 126 (action 488). Determination of measurement 116 may be performed by analysis unit 110/210/310 of biomedical sensing system 100/200/300, using motion compensated diagnostic signal 126 produced by motion correction module 120. As shown in FIGS. 2 and 3E, in some implementations, measurement 116 may be determined and displayed as waveform 216/316. For example, waveform 216/316 may correspond to a cardiac rhythm, fluctuations in blood pressure or blood chemistry, or to any other variable physiological metric of diagnostic interest.

According to the various implementations disclosed herein, one or more motion sensors situated in proximity to one or more diagnostic sensors is/are utilized to sense motion of the diagnostic sensor or sensors as diagnostic sensing is performed. As also disclosed herein, a motion signal received by an analysis unit of a biomedical sensing system from the motion sensor(s) can be used to filter a diagnostic signal corresponding to a physiological metric of a living subject. Such filtering of the diagnostic signal based on the motion signal produces a motion compensated diagnostic signal corrected for the motion of the diagnostic sensor(s) during sensing. The analysis unit may then advantageously determine a measurement of the physiological metric being sensed that is substantially accurate despite the motion of the diagnostic sensor(s) during sensing.

From the above description it is manifest that various techniques can be used for implementing the concepts described in the present application without departing from the scope of those concepts. Moreover, while the concepts have been described with specific reference to certain implementations, a person of ordinary skill in the art would recognize that changes can be made in form and detail without departing from the scope of those concepts. As such, the described implementations are to be considered in all respects as illustrative and not restrictive. It should also be understood that the present application is not limited to the particular implementations described herein, but many rearrangements, modifications, and substitutions are possible without departing from the scope of the present disclosure.

What is claimed is:

1. A biomedical sensing system comprising:
a diagnostic sensor configured to sense a physiological metric of a living subject via a contact with the living subject, and to generate a diagnostic signal corresponding to the physiological metric;
a motion sensor situated proximate the diagnostic sensor, the motion sensor configured to sense a motion corresponding to a motion of the diagnostic sensor during a sensing of the physiological metric by the diagnostic sensor, and to generate a motion signal corresponding to the sensed motion;
an analysis unit including:
a memory storing a motion correction module; and
a processor configured to receive the diagnostic signal and the motion signal, and to execute the motion correction module from the memory to adaptively filter the diagnostic signal using the motion signal to produce a motion compensated diagnostic signal.

2. The biomedical sensing system of claim 1, wherein the motion sensor is in contact with the living subject.

3. The biomedical sensing system of claim 1, wherein the motion sensor is not in contact with the living subject.

4. The biomedical sensing system of claim 1, wherein at least one of the diagnostic signal and the motion signal is transmitted wirelessly.

5. The biomedical sensing system of claim 1, wherein the analysis unit is implemented as part of a personal communication device.

6. The biomedical sensing system of claim 1, wherein the analysis unit is integrated with at least one of the diagnostic sensor and the motion sensor.

7. The biomedical sensing system of claim 1, wherein the analysis unit is further configured to determine a measurement of the physiological metric corresponding to the diagnostic signal based on the motion compensated diagnostic signal.

8. The biomedical sensing system of claim 7, further comprising a display for displaying the measurement of the physiological metric as a waveform corresponding to the physiological metric.

9. The biomedical sensing system of claim 1, further comprising a wearable article including at least one of the diagnostic sensor and the motion sensor.

10. The biomedical sensing system of claim 9, wherein the wearable article includes the diagnostic sensor and the motion sensor.

11. The biomedical sensing system of claim 9, wherein the wearable article includes the analysis unit.

12. The biomedical sensing system of claim 9, wherein the wearable article comprises a patch configured for epidermal attachment to the living subject.

13. The biomedical sensing system of claim 9, wherein the wearable article comprises a cuff configured to encircle a digit of the living subject.

14. The biomedical sensing system of claim 9, wherein the wearable article comprises a band configured to be worn around one of a wrist or an ankle of the living subject.

15. The biomedical sensing system of claim 9, wherein the wearable article comprises a smartwatch.

16. A method for use by a biomedical sensing system including a diagnostic sensor, a motion sensor, and an analysis unit having a processor and a memory, the method comprising:
sensing, using the diagnostic sensor in contact with a living subject, a physiological metric of the living subject;
generating, using the diagnostic sensor, a diagnostic signal corresponding to the physiological metric;
sensing, using the motion sensor, a motion corresponding to a motion of the diagnostic sensor during the sensing of the physiological metric by the diagnostic sensor;
generating, using the motion sensor, a motion signal corresponding to the sensed motion; and
adaptively filtering the diagnostic signal using the motion signal to correct for the motion of the diagnostic sensor during the sensing of the physiological metric, to produce a motion compensated diagnostic signal.

17. The method of claim 16, wherein the motion sensor is in contact with the living subject.

18. The method of claim 16, wherein the motion sensor is not in contact with the living subject.

19. The method of claim 16, further comprising determining, by the analysis unit, a measurement of the physiological metric to which the diagnostic signal corresponds based on the motion compensated diagnostic signal.

20. The method of claim 19, further comprising displaying the measurement of the physiological metric as a waveform corresponding to the physiological metric.
